(19) United States
(12) Patent Application Publication
(10) Pub. No.: US 2020/0057062 A1
(43) Pub. Date: Feb. 20, 2020

(54) DIAGNOSTIC MARKER FOR PREDICTING EFFICACY OF RA DRUG AND APPLICATION THEREOF

(71) Applicant: Peking Union Medical College Hospital, Bejing (CN)

(72) Inventors: Xuan ZHANG, Beijing (CN); Wenxiu MO, Beijing (CN); Yongzhe LI, Beijing (CN); Chaojun HU, Beijing (CN); Guozhen LIU, Beijing (CN); Lijun WU, Beijing (CN)

(21) Appl. No.: 16/485,068

(22) PCT Filed: Nov. 15, 2017
(86) PCT No.: PCT/CN2017/111041

§ 371 Date: Aug. 9, 2019

(30) Foreign Application Priority Data
Feb. 15, 2017 (CN) 201710081288.3

(51) Int. Cl. G01N 33/564

(52) U.S. Cl. CPC G01N 33/564; G01N 2800/52; G01N 2800/102

(57) ABSTRACT

A use of an enhancer of rudimentary homolog (ERH) or a fragment thereof, in the preparation of a reagent for monitoring drug efficacy for rheumatoid arthritis, is provided. 35 proteins as candidate ACPA-negative RA autoantigens, 8 proteins as candidate autoantigens for predicting disease activity, and 6 proteins as candidate autoantigens for predicting treatment effect were screened by hybridizing high density protein chips with RA serum. Of the 6 proteins as candidate autoantigens for predicting treatment effect, one autoantigen, ERH, can successfully determine the effect of drug treatment on RA, and the AUC for predicting efficacy may be up to 0.733.

### TECHNICAL FIELD

The present invention belongs to biological detection field, in particular, relates to a kind of diagnostic marker for predicting the efficacy of RA drug and application thereof.

### BACKGROUND ART

Rheumatoid arthritis (RA) is a chronic autoimmune disease, mainly characterized by the multiple joint inflammation and local bone destruction. In developing countries, rheumatoid arthritis affects nearly 0.5% to 1% of the population. In general, the incidence of RA in women is higher than that in men, and the elderly are more likely to develop RA than the youth. The clinical manifestations of rheumatoid arthritis display heterogeneity, ranging from self-limiting disease with mild symptoms to fast developed inflammation along with joint destruction and severe physical disability. Due to differences in disease performance, classification criteria were developed as the basis for disease definition, selection of standardized clinical trials, and comparison of multicenter studies. In 1987, the classification criteria for RA was established by the American College of Rheumatology (ACR). However, too strict definition for arthritis in the classification criteria leads to a low sensitivity to RA diagnosis in practice, a large number of early RA patients failed to be identified. It is of vital importance that new-onset RA case can benefit from early effective intervention, avoiding progression to chronic and erosive RA or even disability, thus affecting long-term quality of life and increasing disease mortality. Therefore, early diagnosis and treatment serve as the key to preventing irreversible joint destruction. In 2009, the ACR/EULAR standard for RA was updated with higher sensitivity to diagnose and treat RA early, but the specificity still needs improving.

Anti-citrulline polypeptide antibody (ACPAs) positivity is included in the 2009 ACR revised criteria. ACPA is regarded as the serum specific biomarker of RA, the emergence of which improves our understanding of the pathogenesis of RA. But the exact cause of RA is still unknown until now. Environmental and genetic factors are acknowledged as the “trigger” of clinical symptoms in RA. Moreover, there am still a number of ACPA-negative RA patients in clinical practice, and thanks to the researchers' more understanding of the disease, they gradually realize that there is a certain clinical heterogeneity in ACPA-positive RA patients and ACPA-negative RA patients. The lack of specific biomarkers for ACPA-negative RA patients makes it quite difficult to provide accurate diagnosis and treatment.

Autoantibodies have been found in the serum of RA patients for more than 70 years. The rheumatoid factor, targeting the Fc fragment of human IgG, is the first group of autoantibodies identified, including various isotypes such as IgG and IgM. But RF is not the antibody specific to RA since RF can also be detected in many other conditions including the normal elderly, patients with other autoimmune diseases and 15% of healthy individuals. In the years 1964 and 1979, other antibodies, i.e. anti-perinuclear factor antibodies (APF) and anti-keratin antibodies (AKA) were found in RA patients respectively. Although these two antibodies have high specificity in the diagnosis of RA, they are difficult to be detected. RF is still applied for the diagnosis of RA and therefore included in the 1987 ACR RA criteria. Until the year 1995, studies revealed that APF and AKA were similar autoantibodies, both targeting citrulline residue formed by the deimidization of arginine residues. In the year 2002, the first commercialized kit to detect ACPA was developed, enabling ACPA to serve as conventional biomarker for RA. The study of this autoantibody system has deepened our understanding and improved the classification of RA. Therefore, both RF and ACPA have become parts of ACR/EULAR2010 classification criteria.

Recently, it's reported that a novel autoantibody subtype recognizing carbamylated proteins (anti-CarP) was identified in RA patient serum. The autoantibody system is independent of ACPA, since autoantibodies of RA are able to distinguish citrullinated and carbamylated antigens. Correspondingly, anti-CarP antibodies are detected in a part of ACPA-negative patients. In the last few years, the identification of autoantibodies targeting citrullinated and carbamylated proteins helps understand the pathogenesis and etiology of RA. Both carbamylation and citrullination are post-translational modifications, making proteins be carbamylated or citrullinated respectively and replacing positively charged amino acids with the neutral ones. Citrullinated proteins are derived from PAD while Carbamylated proteins are obtained by converting the lysine to homocitrulline by chemical reaction. Citrulline and homocitrulline are chemically similar but located at different sites of the protein because of different location of arginine and lysine. And the homocitrulline has one more formyl group compared to the citrulline. Although there're antibodies reactive to both structures, some anti-CarP antibodies do not respond to citrulline, while some anti-citrulline antibodies also fail to react to CarP. In 2009, Auger et al. tested serum samples of ACPA-negative RA patients with chips containing 8268 proteins and identified PAD4 and BRAF as candidate biomarkers. Anti-PAD antibodies targeting enzymes involving protein citrullination, attracted extensive attention, since it is found that these antibodies can not only bind to targets, but also activate PAD. Anti-PAD antibodies increase the catalytic capacity of PAD4 by decreasing the calcium requirement of this citrullinated enzyme. Charpin C et al. found that there are antibodies against the BRAF catalytic domain in sera from patients with RA, which are mainly focused on amino acids 416 to 766 and the antibodies are present in 30% of ACPA-negative RA patients. Meantime, 33% of patients who are anti-BRAF antibodies positive are ACPA negative. In addition, anti-BRAF antibodies can be found in 4% of AS patients and 6% of healthy controls.

As mentioned above, RA is a chronic autoimmune disease, for which autoantibodies marker detection matters a lot. RA is characterized by clinical heterogeneity. Some patients' symptoms are self-limiting and mild, while some suffer from rapidly progressive inflammation, joint destruction and severe disability. The heterogeneity of RA clinical manifestations leads to great differences of reactions to treatment. For now, there's no way to predict the effect of specific treatment for the lack of efficient biomarkers to sub-classify RA patients. The identification of ACPA is of vital importance since it is the first time to classify RA patients with serum marker. However, ACPA-positive and ACPA-negative RA patients appear to be quite different in genetics of disease and environmental determinants, molecular features of the joint involvement, remission rate and response to therapies. Many ACPA-negative RA patients have limited targets to subclassify, because the lack of potent biomarkers leads to limited targets to classify the clinical manifestation of RA. Identifying more autoantibodies especially for ACPA-negative antibodies can contribute to unraveling the pathogenic role of autoantibodies and the pathogenesis of RA.

In developing countries including China, the incidence and disability rate of RA stay high, making it important to diagnose early and correctly. Identifying more for ACPA-negative RA related autoantibody markers and autoantigens predicting disease activity or therapeutic efficacy, is indispensable to reduce the disability rate of RA.

### SUMMARY OF THE INVENTION

To solve the problems mentioned above, the present invention provides a diagnostic marker for predicting the efficacy of RA drug and application thereof.

The present invention provides use of enhancer of rudimentary homolog (ERH) and their fragments in preparation of reagents for monitoring drug efficacy on rheumatoid arthritis.

In an embodiment of the present invention, monitoring drug efficacy on rheumatoid arthritis includes: detecting the level of the antibody reactive to ERH or their fragments in biological samples from treatment-naive RA patient;

If there are antibodies reactive to ERH or its fragments in biological samples, it can be predicted that the patient will achieve moderate remission or above after taking drugs regularly for 3 to 6 months. If there is no antibody reactive to ERH or its fragments in the biological samples, it can be predicted that the patient will not be able to achieve effective remission after 3 to 6 months of regular medication. wherein, the biological samples refer to serum samples.

Wherein, the said drugs may be any drug used in the field for treating or relieving rheumatoid diseases, preferably from low-dose corticosteroids and/or traditional disease-modifying anti-rheumatic drugs (DMARDs).

In the specific embodiment of the present intervention, the antibody level of the ERH is detected by procedures below, including:

a. making biological samples from patients contact with ERH and their fragments;
b. making the antibodies in the biological sample and ERH or their fragments to form an antibody-protein complex;
c. washing to remove any antibodies uncombined;
d. adding labeled antibodies reactive to antibodies from the biological sample;
e. washing to remove labeled detection antibodies that are uncombined; and
f. transforming the marker of the detection antibodies into detectable signal; wherein the presence of detectable signal indicates the presence of anti-ERH antibodies in patients.
wherein, ERH or their fragments are deposited or fixed in solid phase support.
wherein, the solid support refers to forms of latex beads, porous flat plate or membranes.
wherein, the detection antibodies are labeled by markers that are covalently linked to the enzyme and have fluorescent compounds or metal, or chemiluminescent compounds.

By hybridizing the high density protein chip with RA serum, the present invention identified 35 candidate ACPA-negative RA autoantigens with the specificity more than 90% and the sensitivity more than 25%, and 7 candidate autoantigens associated with prediction of the disease activity and 6 candidate autoantigens associated with prediction of therapy efficacy (two candidate autoantigens are included in the analysis of different groups). To validate the sensitivity and specificity of these autoantigens, a protein chip containing 46 candidate RA autoantigens was constructed. A large number of serum samples (including serum from 9 OA, 38 SLE, 39 AS, 18 BD, 10 ANCA, 21SS, 102 healthy controls and 290 RA) are hybridized with autoantigen chip. Data shows that 4 proteins are newly-identified antigens with high sensitivity and specificity: DOHH, DUSP11, PTX3, PAGE5, and the sensitivity of DOHH and PTX3 as diagnostic markers are 49.66% and 43.54% respectively. In the 7 candidate autoantigens related to prediction of the disease activity, autoantigen RRN3 can distinguish moderate to low activity and high activity in RA successfully and its AUC reaches 0.65. RRN3 and PLEKHG2 can distinguish moderate to low activity and high activity in ACPA-positive RA successfully: the AUC are 0.845 and 0.817, respectively. In 6 candidate autoantigens to predict therapeutic efficacy, ERH can predict the therapeutic efficacy, with the AUC of 0.733.

### DESCRIPTION OF DRAWINGS

FIG. 1: Quality control of protein chips.

FIG. 2: The correlation of all recombinant protein probe parallel points on the protein chips by GST.

FIG. 3: Partial images formed by hybridizing high-density protein chip with small number of serum samples.

FIG. 4: Signal value distribution of Blank and EMPTY on protein chips.

FIG. 5: Signal value distribution of PTX3 in RA patients, healthy and diseases control groups.

FIG. 6: Signal value distribution of RRN3 in different disease activity group.

FIG. 7: Signal value distribution of two antigens in ACPA-positive RA with different disease activity.

FIG. 8: Signal value distribution of ERH in RA group with different efficacy and the AUC curve.

### DETAILED DESCRIPTION OF EMBODIMENTS

The following examples are described to illustrate the present invention, but not limit the scope of the present invention.

Serum samples (all samples were collected and clinically tested at department of rheumatology, Peking Union Medical College Hospital)

The study used 647 serum samples including:

350 samples from RA patients, age (mean±SD): 45.2±12.5
9 samples from osteoarthritis (OA) patients, age (mean±SD): 67.2-16.6
48 samples from systemic lupus erythematosus (SLE) patients, age (mean±SD): 36.8±12.4
28 samples from Beheet's disease (BD) patients, age (mean±SD): 54.2±20.7
10 samples from antineutrophil cytoplasmic antibody-associated vasculitis patients, age (mean±SD): 46.9±16.3
39 samples from ankylosing-spondylitis (AS) patients, age (mean±SD): 38.2±15.1
21 samples from Sjogren Syndrome (SS) patients, age (mean±SD): 52.7-13.2
10 samples from Takayasu arteritis (TA) patients, age (mean±SD): 38.4-13.5
132 samples from healthy controls, age (mean±SD): 37.5±12.1

RA was diagnosed according to the 2010 ACR/EULAR criteria for RA, and OA, SLE, BD, ANCA, AS, SS and TA were diagnosed according to corresponding diagnosis and/or criteria.

All serum samples were collected at Peking Union Medical College Hospital during a period from 2006 to 2014. All serum samples are from patients confirm diagnosis at clinic and for those with controversial diagnoses, three chief physicians were invited to identify the final diagnosis.

All RA serum samples were tested for corresponding antibodies, including three ANAs: ANA-IF (immunofluorescence method), DNA-IF (immunofluorescence method), ds-DNA (ELISA), anti-CCP antibodies, i.e. ACPA (positive: >25 IU/ml), RF, AKA and APF, MCV, and GPI. All the anti-CCP antibodies and/or anti-AKA/APF/MCV antibody-negative RA patients satisfy the diagnostic criteria of ultrasound or MRI about RA synovitis. The study was approved by ethics committees of Peking Union Medical College Hospital Review Board.

### Example 1 Identification of Candidate RA Autoantigens Using High-Density Protein Chips

The high-density protein chips and Saccharomyces cerevisiae-expressing recombinant vectors including target gene sequences were provided by Dr. Zhu's laboratory at Johns Hopkins University. Each chip consisted of 48 blocks and the block included 992 probe points arranged in a 32*31 array, with 2 parallel points for each protein probe. The protein chip consisted of 21827 non-redundant recombinant human proteins. The recombinant proteins, with glutathione S-transferase (GST) tag at the N-terminus, were derived from the full-length open reading frame (ORF) of the corresponding gene expressed by Saccharomyces cerevisiae.

The quality of chips was verified by hybridizing mouse anti-GST monoclonal antibodies with the chips. Qualified repeatability of duplicate protein spots was achieved when the correlation coefficient of fluorescent signal value between duplicate spots reached 97%.

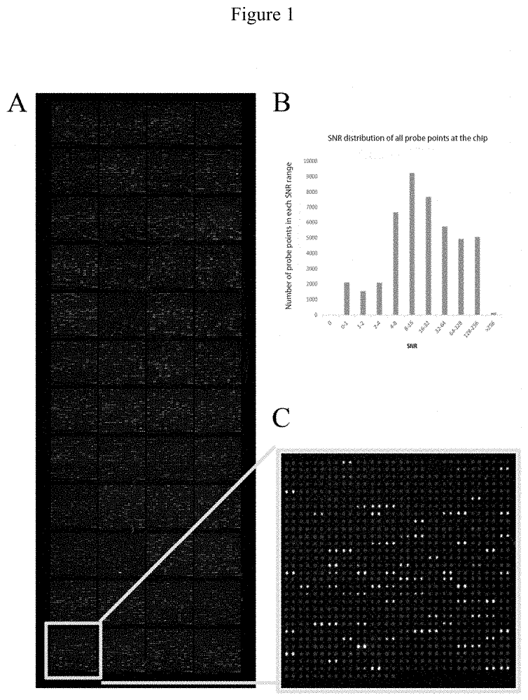

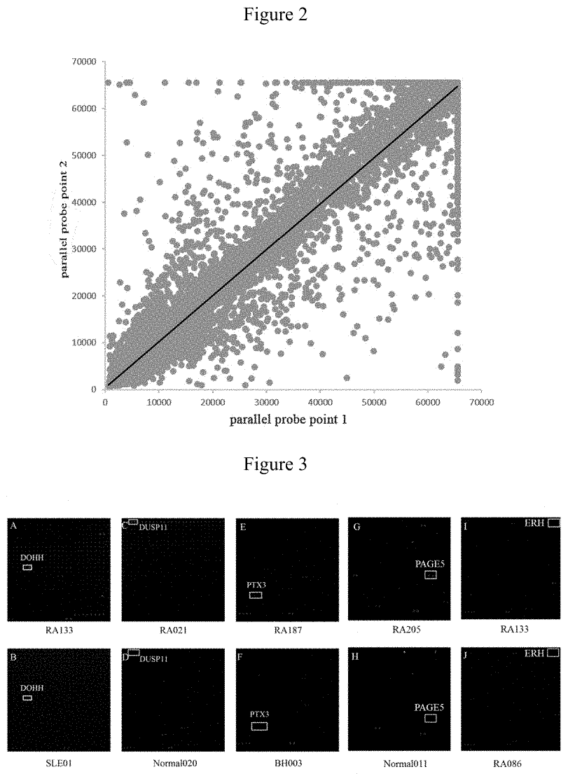

Each high-density protein chip included 47616 protein spots (including positive control and negative control; each protein antigen included two parallel points). The chip consisted of 21827 non-redundant recombinant human proteins. All proteins on each chip consisted of 48 blocks and each block was arranged in a 32*31 array. For all the recombinant protein probes carried GST tag at the N-terminus, mouse anti-GST monoclonal antibodies were used for detection of all probes at the chip, in order to make sure that the majority of recombinant proteins at the chip for serum identification at the chip were detectable and two parallel points on the same probe had high parallelism. As shown in FIG. 1, GST tag-positive points detected at the chip appear to be red (white when the signal is saturated). Each protein chip consisted of 48 blocks and all protein probes in each block was arranged in a 32*31 array, and each probe consisted of two parallel points (left point and right point). Each chip consisted of 21827 non-redundant recombinant proteins and other control probes. All recombinant proteins carried GST tag. FIGS. 1A and 1C show the scan image of the whole chip and single block respectively, using mouse anti-GST monoclonal antibodies for detection. FIG. 1B shows the distribution of all probes' signal to noise ratio (SNR). The probe point was considered to be detectable when the SNR of two parallel points was greater than 3. According to the standard, 96.8% of the proteins were detectable (FIG. 2).

### Example 2 Hybridization of High-Density Protein Chips, RA and Control Serum

60 RA and 60 control (10 BD, 10 TA, 10 SLE and 30 healthy controls) serum samples were hybridized with 120 protein chips, and candidate RA autoantigens were identified by signal collection and data analysis. PE-Cy5 labeled anti-human IgG antibody was used to detect the reaction between the serum autoantibodies and autoantigen probes. FIG. 3 shows the representative partial scan image formed by serum hybridization with high-density protein chips, different protein antigen probe is shown in the box. A, C, E, G show scan images of hybridization of 4 RA serum samples with chips. B, D, F, H show scan images of hybridization of 4 control serum samples (including disease and healthy controls) with chips. FIG. 1 shows the scan image of RA treatment effective. Figure J shows the scan image of RA treatment ineffective. Two parallel points protein probes in the boxes of Figures A and B are DOHH, DUSP11 in the boxes of Figures C and D, PTX3 in the boxes of Figures E and F, PAGE5 in the boxes of Figures G and H, ERH in the boxes of Figures I and J. In general, serum from RA, disease control (BD, SLE, TA), or healthy group only recognizes a small part of proteins at the chip. And positive signal can also be detected in chips of normal control serum, suggesting autoantibodies can exist in healthy group, but these autoantibodies do not lead to diseases.

The fluorescence signal images of each chip obtained by scan and the template file of the chip, i.e. gail files were dragged to GenePix Pro 6.0 for one to one correspondence. All probe signal information of each chip collected by GenePix Pro 6.0 was transformed and saved in excel format The signal value was calculated by the ratio of foreground (F635 median) to background (B635 median) signals. i.e. I ij=F635 median B635 median (I ij represented the signal value of protein point i in block j). As the protein antigen probe signal value was closer to 1, the corresponding autoantibody in serum became less detectable. Higher signal value meant the stronger ability of autoantibodies to bind the target protein antigen probe.

To eliminate the hybridization differences caused by different chips or different space in the same chip, within-chip normalization was adopted to normalize the intra-array signal intensity, which means we hypothesized that all target proteins were placed on the substrate at random, and only less 5% of target proteins as autoantigens were identified and detected by corresponding target antibody. Consequently, the signal distribution at the chips was random and remained consistent between different blocks. The median of all the probe point signal value was set as 1 to normalize the probe point signal value in different blocks. Ĩ ij=I ij−median(Ij)+1 (median(Ij) represented the median of all points signal value in the block j, Ĩ ij represented the signal value of protein point i in the block j after normalization.

On this basis, according to the method of paper (Hu S, Xie Z, Onishi A, Yu X, Jiang L, Lin J, Rho H S, Woodard C, Wang H, Jeong J S, Long S, He X, Wade H, Blackshaw S, Qian J, Zlu H, Profiling the human protein-DNA interactome revealed ERK2 as a transcriptional repressor of interferon signaling. Cell 2009; 139: 610-622), the cutoff was set to identify the positivity of all probe points, i.e. calculating the mean of all point signal value at whole chip (I average), setting the standard deviation (SD) of signal values less than 1; setting I average +5SD as cutoff to analyze the positivity of probe points at the chip; collecting the positivity information of immune reaction between each serum with each protein antigen probe, and using chi-square test (X2) or Fisher exact test to determine the candidate RA autoantigen.

For the identification of ACPA-negative RA candidate markers, antigens with specificity more than 90% and sensitivity more than 25% served as candidate RA autoantigens. For the identification of candidate biomarkers predicting disease activity and treatment efficacy, if P<0.05 (calculated by chi-square test or Fisher exact test), it will be included in candidate markers.

The candidate target autoantigens of interest at the chip were determined by data analysis. For whether the on-chip protein probe was a RA-specific autoantigen, or whether it is a disease-associated or therapeutically relevant autoantigen, the X2 test or Fisher's exact test was used to determine that the protein was a target protein antigen for the ACPA-negative specific reaction in RA. In the present invention, 35 antigens with a specificity of 90% and a sensitivity of more than 25% were used as candidate ACPA-negative RA autoantigens, and 7 proteins were candidate autoantigens for predicting disease activity, and 6 proteins were candidate autoantigens for predicting therapeutic efficacy (wherein two protein candidate antigens were repeated in different sets of analyses), see Table 1 for details.

TABLE 1-1
Small sample sera were hybridized with high-density protein chips to identify 35 candidate ACPA-negative RA-specific autoantigens

| | | | | Number of | Number of | Number of | Number of | Number of |
|---|---|---|---|---|---|---|---|---|
| | | | | positive | positive | positive | positive | positive |
| | | | | probes | probes | probes | probes | probes |
| | | | | identified | identified | identified | identified | identified |
| Abbreviation | | | | in 30 CCP- | in 30 CCP- | in 60 RA | in 30 disease | in 30 healthy |
| of name of | | | | RA serum | RA serum | serum | control | control |
| proteins | ID No. | Antigen | Name of proteins | group | group | group | group | group |
| DOHH | NM_031304.2 | newly | Deoxyhypusine dioxygenase | 9 | 10 | 19 | 1 | 2 |
| | | identified | | | | | | |
| DUSP11 | BC000346.2 | newly | Dual specificity protein | 11 | 12 | 23 | 0 | 0 |
| | | identified | phosphatase 11 | | | | | |
| PTX3 | NM_002852.3 | newly | Pentaxin-related protein PTX3 | 12 | 13 | 25 | 0 | 1 |
| | | identified | | | | | | |
| STK3 | NM_006281.2 | newly | Serine/threonine- protein | 13 | 20 | 33 | 0 | 1 |
| | | identified | kinase Krs-1 | | | | | |
| HDAC4 | BC039904.1 | newly | Histone deacetylase 4 | 12 | 19 | 31 | 0 | 1 |
| | | identified | | | | | | |
| RAI14 | BC052988.1 | newly | Ankyrin repeat and coiled-coil | 12 | 19 | 31 | 0 | 1 |
| | | identified | structure-containing protein | | | | | |
| FGF12 | BC022524.1 | newly | Fibroblast growth factor | 11 | 16 | 27 | 0 | 2 |
| | | identified | homologous factor 12 | | | | | |
| RAB35 | NM_006861.4 | newly | Ras-related protein Rab-35 | 12 | 15 | 27 | 0 | 1 |
| | | identified | | | | | | |
| APH1A | NM_016022.2 | newly | Gamma-secretase subunit | 10 | 15 | 25 | 0 | 0 |
| | | identified | APH-1A | | | | | |
| CHAC2 | NM_001008708.1 | newly | Putative glutathione-specific | 14 | 14 | 28 | 0 | 1 |
| | | identified | gamma-glutamylcyclo- | | | | | |
| | | | transferase 2 | | | | | |
| ND | BC000566.2 | newly | Homo sapiens RAB, | 14 | 14 | 28 | 0 | 2 |
| | | identified | member of RAS | | | | | |
| | | | oncogene family-like 4 | | | | | |
| TBC1D19 | ENST00000264866 | newly | TBC1 domain family | 12 | 14 | 26 | 0 | 2 |
| | | identified | member 19 | | | | | |
| NECAB1 | NM_022351.2 | newly | N-terminal EF-hand calcium- | 9 | 14 | 23 | 2 | 1 |
| | | identified | binding protein 1 | | | | | |
| AK2 | NM_001625.2 | newly | Adenylate kinase 2 | 12 | 13 | 25 | 0 | 0 |
| | | identified | | | | | | |
| ERH | NM_004450.1 | newly | Enhancer of rudimentary | 11 | 13 | 24 | 0 | 2 |
| | | identified | homolog | | | | | |
| ATP13A5 | NM_193505 | newly | Probable cation-transporting | 14 | 12 | 26 | 1 | 2 |
| | | identified | ATPase 13A5 | | | | | |
| PAGE5 | NM_001013435.1 | newly | P antigen family member 5 | 11 | 12 | 23 | 1 | 2 |
| | | identified | | | | | | |
| SUGT1 | NM_006704.2 | newly | Suppressor of G2 allele of | 11 | 12 | 23 | 1 | 2 |
| | | identified | SKP1 homolog | | | | | |
| MYLK | NM_053031.2 | newly | Myosin light chain kinase | 10 | 12 | 22 | 2 | 2 |
| | | identified | | | | | | |
| NDRG1 | NM_006096.2 | newly | N-myc downstream-regulated | 9 | 12 | 21 | 0 | 0 |
| | | identified | gene 1 protein | | | | | |
| CHST11 | NM_018413.2 | newly | Carbohydrate sulfotransferase 11 | 9 | 12 | 21 | 0 | 1 |
| | | identified | | | | | | |
| STK24 | BC065378.1 | newly | Serine/threonine- protein | 9 | 12 | 21 | 1 | 1 |
| | | identified | kinase 24 | | | | | |
| ND | NM_002720.1 | newly | Homo sapiens protein | 9 | 11 | 20 | 0 | 0 |
| | | identified | phosphatase 4 | | | | | |
| | | | catalytic subunit (PPP4C) | | | | | |
| RAB3D | NM_004283.2 | newly | Ras-related protein Rab-3D | 9 | 11 | 20 | 0 | 0 |
| | | identified | | | | | | |
| POLR3B | BC046238.1 | newly | Homo sapiens polymerase | 9 | 10 | 19 | 0 | 0 |
| | | identified | (RNA) III polypeptide B, | | | | | |
| | | | mRNA | | | | | |
| UBXN10 | NM_152376.2 | newly | UBX domain-containing | 9 | 10 | 19 | 0 | 1 |
| | | identified | protein 10 | | | | | |
| ATXN10 | BC007508 | newly | Spinocerebellar ataxia type 10 | 9 | 10 | 19 | 1 | 1 |
| | | identified | protein | | | | | |
| TNFAIP1 | NM_021137.3 | newly | Tumor necrosis factor, alpha- | 10 | 9 | 19 | 2 | 0 |
| | | identified | induced protein 1, endothelial | | | | | |
| METTL21C | NM_001010977.1 | newly | Protein-lysine methyltransferase | 9 | 9 | 18 | 0 | 1 |
| | | identified | METTL21C | | | | | |
| NDEL1 | BC026101.2 | newly | Nuclear distribution protein | 9 | 9 | 18 | 1 | 1 |
| | | identified | nudE-like 1 | | | | | |
| LSP1 | BC001785.1 | newly | Lymphocyte-specific protein 1 | 9 | 9 | 18 | 2 | 1 |
| | | identified | | | | | | |
| BABAM1 | NM_001033549.1 | newly | BRISC and BRCA1-A complex | 11 | 7 | 18 | 0 | 1 |
| | | identified | member 1 | | | | | |
| DGKK | NM_001013742.2 | newly | Diacylglycerol kinase kappa | 10 | 7 | 17 | 2 | 2 |
| | | identified | | | | | | |
| PPFIA4 | NM_015053.1 | newly | Protein tyrosine phosphatase | 9 | 7 | 16 | 2 | 0 |
| | | identified | receptor type f polypeptide- | | | | | |
| | | | interacting protein alpha-4 | | | | | |
| PDCD2 | NM_002598.2 | newly | Programmed cell death protein 2 | 9 | 7 | 16 | 0 | 1 |
| | | identified | | | | | | |

TABLE 1-2
Small sample serum were hybridized with high-density protein chip to
identify 7 candidate autoantigens for predicting disease activity.

| | | Number of positive |
|---|---|---|
| | Number of positive | probes identified |
| | probes identified | to 20 moderate to |
| | to 40 high activity | low activity RA |
| Name of proteins | RA serum group | serum group |
| Zinc finger and SCAN | 9 | 11 |
| domain-containing protein 20 | | |
| Protein FAM84A (Neurologic | 7 | 9 |
| sensory protein 1) (NSE1) | | |
| Sorting nexin-33 (SH3 and | 8 | 9 |
| PX domain-containing protein | | |
| 3) | | |
| RNA polymerase I-specific | 5 | 8 |
| transcription initiation | | |
| factor RRN3 | | |
| Pleckstrin homology domain- | 2 | 6 |
| containing family G member | | |
| 2 | | |
| Nucleolar protein 3 | 3 | 6 |
| RING finger protein 183 | 13 | 2 |

TABLE 1-3
Small sample serum were hybridized with high-density
protein chip to identify 7 candidate autoantigens
for predicting disease therapeutic efficacy

| | Number of positive | Number of positive |
|---|---|---|
| | probes identified | probes identified |
| | to 20 RA serum group | to 20 RA serum group |
| | without therapeutic | with therapeutic |
| Name of proteins | efficacy | efficacy |
| Regulator of cell cycle | 1 | 9 |
| RGCG | | |
| Tumor necrosis factor, | 7 | 4 |
| alpha-induced protein 1, | | |
| endothelial | | |
| Glycine-tRNA ligase | 8 | 4 |
| Sperm protein associated | 7 | 3 |
| with the nucleus on the | | |
| X chromosome N2 | | |
| ADP-ribosylation factor- | 7 | 3 |
| like protein 2 binding | | |
| protein | | |
| Enhancer of rudimentary | 8 | 14 |
| homolog | | |

### Example 3 Construction of RA Autoantigen Protein Chip and Verification of Serum Screening

By analyzing the result of hybridizing small number of serum samples with high-density protein chips, 46 candidate RA autoantigens were identified. To verify the specificity and sensitivity of these autoantigens, the present invention constructed low probe density RA autoantigen protein chip. Table 2 shows the distribution of microarray of each probe at RA autoantigens protein chip. The probes at the chip included 46 candidate RA autoantigens and 5 control probes (IGHG1).

TABLE 2
Microarray of each probe at RA autoantigens protein chip

| | | | | | |
|---|---|---|---|---|---|
| AK2 | IGHG1 | ND | ATP13A5 | ND | TBC1D19 |
| RAB35 | UBXN10 | RAB3D | APH1A | TNFAIP1 | HDAC4 |
| ARL2BP | RAI14 | RRN3 | POLR3B | ERH | NDRG1 |
| BLANK | BLANK | BLANK | BLANK | BLANK | GARS |
| SUGT1 | IGHG1 | NOL3 | ZSCAN20 | LSP1 | RGCC |
| EMPTY | PAGE5 | FGF12 | FAM84A | DOHH | NECAB1 |
| NDEL1 | DUSP11 | PDCD2 | MYLK | STK24 | METTL21C |
| IGHG1 | STK3 | BABAM1 | DGKK | PTX3 | PPFIA4 |
| EMPTY | SPANXN2 | IGHG1 | CHAC2 | RNF183 | ATXN10 |
| IGHG1 | EMPTY | CHST11 | PLEKHG2 | SNX33 | BLANK |

51 probes at RA autoantigen protein chip all had duplicate points. 14 microarrays were included at each substrate and every microarray was separated by the fence to form independent space before hybridization of serum and chip. Every chip can detect 14 serum samples at the same time. The large scale samples of serum hybridized with RA autoantigen chip included 290 RA, and 237 controls serum (9 OA, 38 SLE, 39 AS, 18 BD, 10 ANCA, 21 SS and 102 healthy controls serum). Genepix Pro6.0 was applied to acquire the information of probe points from the hybridization of RA autoantigen protein chip. The signal strength was calculated by the ratio of foreground to background signals of each probe point. The average of two parallel points hybridization signal of each probe was set as the signal value of hybridization of the probe and the serum for further analysis.

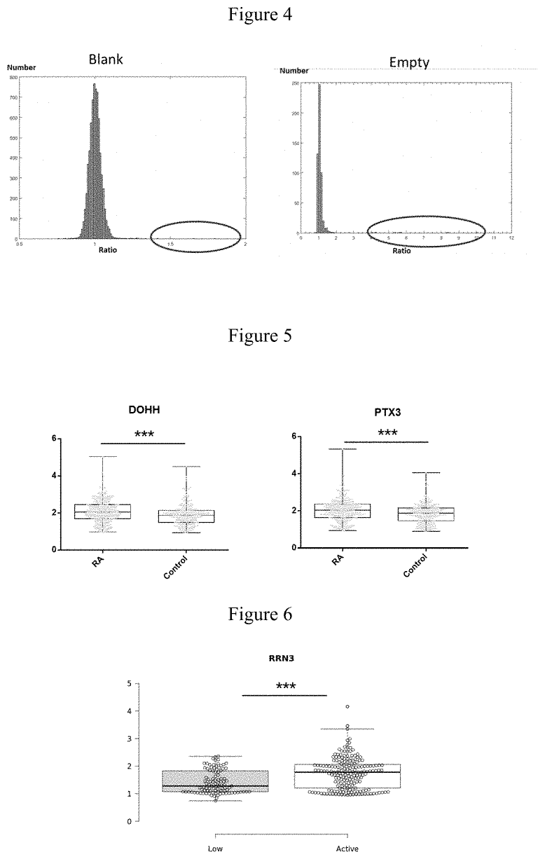

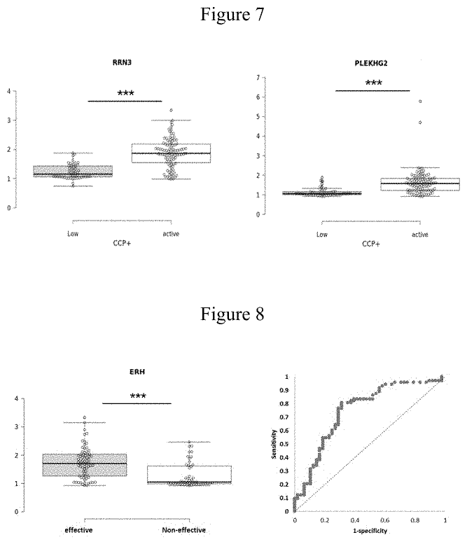

Negative control protein pore signal was used to evaluate the quality of experiments. The prepared protein chip consisting of 46 candidate RA autoantigens included 6 blank control (BLANK) and 3 negative control (EMPTY). The average of negative control protein pore signal values was used for the quality assessment of the protein chip. The signal value of each block's negative control protein on each chip was collected for drawing a frequency distribution of signal values. As shown in FIG. 4, the signal value of the BLANK and EMPTY was around 1, indicating that the foreground value and background value of the point are nearly identical and signal values collected from the chips were rational and reliable.

Chi-square test (X2) or Fisher exact test were used to analyze data from ACPA-negative RA patients and healthy and disease controls, calculating T score, P value of every diagnostic marker protein. 1000 different cutoff values were selected for each protein, specificity and sensitivity can be calculated according to each cutoff value, these 1000 points (1-specificity, sensitivity) were used to draw the ROC curve, and the AUC was calculated. The cutoff that corresponded to the point with the maximum sum of sensitivity and specificity was the best cutoff. As shown in Table 3 and FIG. 5, in the hybridization results with large scale samples of serum, the sensitivity of immune reaction of four protein antigens with ACPA-negative RA serum was higher than 25%, and these four protein antigens had specificity different from healthy controls and disease controls (DOHH: 49.66%, PAGE5: 72.79%, DUSP11: 53.06%, PTX3: 43.54%). FIG. 5 shows the distribution of signal value of these two protein markers in RA patients and healthy controls and disease controls, indicating that autoantibodies expression in RA patients is higher than the controls.

TABLE 3
The cutoff and corresponding AUC of four proteins including DOHH in RA

| Name | T Score | p value | FDR(BH) | Q Value | Fold Change | AUC | cutoff | Specificity | Sensitivity |
|---|---|---|---|---|---|---|---|---|---|
| PAGE5 | −3.49914 | 6.00E−04 | 0.005056 | 0.001438 | 1.11134349 | 0.627525 | 1.830343 | 0.487437 | 0.727891 |
| PTX3 | −3.37216 | 6.00E−04 | 0.005056 | 0.001438 | 1.13017941 | 0.594742 | 2.104885 | 0.743719 | 0.435374 |
| DOHH | −2.23377 | 0.020596 | 0.050632 | 0.01337 | 1.08083119 | 0.599221 | 2.02812 | 0.693467 | 0.496599 |
| DUSP11 | −1.79863 | 2.00E−04 | 0.002949 | 0.001038 | 1.24243701 | 0.612484 | 2.112003 | 0.668342 | 0.530612 |

### Example 4 Analysis of the Newly-Identified Antigens for Predicting Disease Activity in RA Autoantigen Protein Chip

The T test was used to analyze data of two groups of patients with moderate to low activity and high activity, calculating T score, P value for each protein associated with predicting disease activity. Then 1000 different cutoff values were selected for each protein, specificity and sensitivity can be calculated according to each cutoff value, these 1000 points (1-specificity, sensitivity) were used to draw the ROC curve, and the AUC was calculated. The cutoff that corresponded to the point with the maximum sum of sensitivity and specificity was the best cutoff. As shown in Table 4 and FIG. 6, the AUC of RRN3 is highest, 0.65, when the cut off is 1.55. FIG. 6 shows the signal value distribution of groups with moderate to low activity and high activity, patients with high activity expressing more autoantigen than patients with moderate to low activity.

TABLE 4
The cutoff and corresponding AUC of RRN3 in RA patients with different disease activity

| | | | | | Fold | active | active | low | low | | | | |
|---|---|---|---|---|---|---|---|---|---|---|---|---|---|
| Name | Score | P | FDR(BH) | Q Value | Change | Mean | Std | Mean | Std | Cutoff | AUC | Sensitivity | Specificity |
| RRN3 | 5.49 | 2.00E−04 | 6.55E−04 | 0.000197 | 1.23 | 1.74 | 0.564 | 1.42 | 0.42 | 1.55 | 0.65 | 64.10% | 69.40% |

Further analysis of the clinical sub-group revealed that autoantigen PLEKHG2 could distinguish disease activity in ACPA-positive RA patients, but not ACPA-negative RA patients. When the cutoff was 1.548 for RRN3 and 1.172 for PLEKHG2, and the corresponding AUC was 0.845 and 0.817 respectively, indicating the great clinical value.

TABLE 5
The cutoff and AUC of RRN3 and PLEKHG2 in different disease activity group of ACPA-positive patients

| | | | | | | CCP + | CCP + | ccp + | ccp + | | | | |
|---|---|---|---|---|---|---|---|---|---|---|---|---|---|
| | | | | | Fold | low | low | active | active | | | Speci- | Sensi- |
| Name | Score | P | FDR(BH) | Q Value | Change | Mean | Std | Mean | Std | Cutoff | AUC | ficity | tivity |
| RRN3 | −10.18 | 2.00E−04 | 5.62E−04 | 0.044524 | 1.522 | 1.237 | 0.254 | 1.883 | 0.504 | 1.548 | 0.845 | 91.70% | 74.70% |
| PLEKHG2 | −6.83 | 2.00E−04 | 5.62E−04 | 0.155012 | 1.444 | 1.132 | 0.21 | 1.634 | 0.652 | 1.172 | 0.817 | 79.20% | 82.10% |

### Example 5 Analysis of the Newly-Identified Antigens for Predicting Disease Therapeutic Efficacy in RA Autoantigen Protein Chip

T test was used to analyze data from effective and non-effective RA patients, calculating T score, P value for each protein associated with predicting disease therapeutic efficacy. Then 1000 different cutoff values were selected for each protein, specificity and sensitivity can be calculated according to each cutoff value, these 1000 points (1-specificity, sensitivity) were used to draw the ROC curve, and the AUC was calculated. The cutoff that corresponded to the point with the maximum sum of sensitivity and specificity was the best cutoff. As shown in Table 6 and FIG. 8, when the cutoff was set at 1.201, the AUC was the largest, 0.733. FIG. 8 shows the signal value distribution of ERH in effective and non-effective patients, indicating that the autoantigen expressed in the effective patients are more than the non-effective patients.

TABLE 6
The cutoff and AUC of ERH in different efficacy group of RA patients

| | | | | | | | effec- | effec- | non- | non- | | | | |
|---|---|---|---|---|---|---|---|---|---|---|---|---|---|---|
| | | | | | | Fold | tive | tive | effective | effective | | | Sensi- | Speci- |
| Name | Description | Score | P | FDR(BH) | Q Value | Change | Mean | Std | Mean | Std | Cutoff | AUC | tivity | ficity |
| ERH | NM_004450.1 | 4.86 | 2.00E−04 | 0.003933 | 0.005899 | 1.341 | 1.733 | 0.545 | 1.292 | 0.446 | 1.201 | 0.733 | 80.8% | 68.7% |

The description above is only a preferred practice of the present invention, and it should be noted that the skilled person in the art can make improvements and modifications without departing from the technical principles of the present invention. These improvements and modifications should also be considered as the scope of protection of the present invention.

1. (canceled)

2. A method of monitoring drug efficacy in treatment of rheumatoid arthritis, comprising:
detecting a level of an antibody to ERH or a fragment thereof in a biological sample from a treatment-naive RA patient; wherein
the presence of antibodies reactive to ERH or fragments thereof indicates that the patient will achieve at least a moderate remission after taking a drug regularly for 3 to 6 months, and the absence of an antibody reactive to ERH or its fragments thereof indicates that the patient will not be able to achieve effective remission after 3 to 6 months of taking a drug.

3. The method as claimed in claim 2, wherein said biological sample is a serum sample.

4. The method as claimed in claim 2, wherein said drug is selected from the group consisting of low-dose corticosteroids and traditional disease-modifying anti-rheumatic drugs.

5. The method as claimed in claim 2, wherein the level of the anti-ERH antibody is detected by:
a. contacting a biological sample from a patient with ERH or a fragment thereof,
b. forming an antibody-protein complex,
c. washing to remove any unbound antibodies,
d. adding labeled detection antibodies reactive to antibodies from the biological sample,
e. washing to remove unbound labeled detection antibodies, and
f. transforming a marker of the detection antibodies into a detectable signal, wherein the presence of the detectable signal indicates the presence of anti-ERH antibodies.

6. The method as claimed in claim 5, wherein, said ERH or fragments thereof are deposited or fixed onto a solid phase support.

7. The method as claimed in claim 6, wherein the solid support is selected from the group consisting of latex beads, porous flat plate and membranes.

8. The method as claimed in claim 5, wherein, the detection antibodies are labeled with markers that are covalently linked to an enzyme and comprise fluorescent compounds or metal, or chemiluminescent compounds.

9. (canceled)

10. (canceled)

11. (canceled)
